(19) United States
(12) Patent Application Publication
(10) Pub. No.: US 2024/0417682 A1
(43) Pub. Date: Dec. 19, 2024

(54) METHODS AND SYSTEMS FOR CULTURING ORGANOIDS

(71) Applicants: NATIONAL STEM CELL FOUNDATION, Louisville, KY (US); NEW YORK STEM CELL FOUNDATION, INC., New York, NY (US)

(72) Inventors: Scott A. NOGGLE, New York, NY (US); Valentina FOSSATI, New York, NY (US); David MAROTTA, New York, NY (US); Jeanne LORING, La Jolla, CA (US)

(21) Appl. No.: 18/700,180

(22) PCT Filed: Oct. 14, 2022
(86) PCT No.: PCT/US2022/046728

§ 371 Date: Apr. 10, 2024

Related U.S. Application Data
(60) Provisional application No. 63/256,279, filed on Oct. 15, 2021.
(60) Provisional application No. 63/344,777, filed on May 23, 2022.

(51) Int. Cl. C12N 5/079; C12M 1/00; C12M 3/00

(52) U.S. Cl. CPC C12N 5/0618; C12M 21/08; C12M 29/06; C12N 2500/02; C12N 2501/13; C12N 2501/22; C12N 2501/2334; C12N 2503/04; C12N 2513/00

(57) ABSTRACT

A method of culturing an organoid includes combining a neural precursor cell with a first volume of a cortical media or a dopaminergic media to form an organoid. A microglia is then added to the organoid, and the combined organoid and microglia are added to a cryovial with a second volume of the cortical media or the dopaminergic media, and which includes an amount of IL-34 and GM-CSF and an amount of a buffering solution. The cryovial is subsequently sealed and the organoid is cultured for a pre-determined period of time under closed conditions. Systems for culturing an organoid include an organoid formed from a neural precursor cell, a microglia, an effective amount of a cortical media or a dopaminergic media, a buffering solution, and a cryovial for housing the aforementioned components under closed conditions.

### RELATED APPLICATIONS

This application claims priority from U.S. Provisional Application Ser. No. 63/256,279, filed Oct. 15, 2021, and U.S. Provisional Application Ser. No. 63/344,777, filed May 23, 2022, the entire disclosures of which are incorporated herein by this reference.

### TECHNICAL FIELD

The presently-disclosed subject matter generally relates to methods and systems for culturing an organoid. In particular, certain embodiments of the presently-disclosed subject matter relate to methods and systems for culturing an organoid under closed conditions and/or microgravity conditions for an extended period of time.

### BACKGROUND

Humans in space face many challenges that alter biological processes and induce adaptation to the new environment. While studies have focused on the effects of microgravity on musculoskeletal degeneration, there is little information about its effect on the human brain. Evidence suggests that the effects of microgravity on astronauts may in some ways parallel age-related illnesses. For example, microgravity accelerates protein misfolding and accumulation, both hallmarks of neurodegenerative disease. Additionally, the astronaut studies such as the NASA twin study revealed microgravity-associated increases in peripheral inflammatory cytokines and chemokines, and upregulation of immune-related pathways during spaceflight. As neuroinflammation contributes to the pathogenesis and progression of many neurodegenerative disorders, understanding the impact of inflammation on the brain in microgravity may prove important to unravelling disease mechanisms and develop sustainable strategies to expand space exploration.

With an increasing average human lifespan, the prevalence of neurodegenerative diseases is on the rise worldwide. Current medications can only manage and improve some symptoms but do not stop, slow, or reverse the neuronal degeneration and loss that occurs during disease progression. Such neurodegenerative diseases include Parkinson's disease (PD) and progressive multiple sclerosis (MS), for which effective treatments are still lacking.

Among the pathogenic mechanisms leading to neurodegeneration in MS and PD, neuroinflammation triggered by glial cells is increasingly recognized as an important factor. Astrocytes, microglia, and oligodendrocytes are non-neuronal cells of the central nervous system (CNS) that are essential to maintain CNS homeostasis. Altered functions of astrocytes and microglia have been directly or indirectly correlated to neuronal degeneration. For example, microglia participate in defense mechanisms, migrating toward injury sites, releasing cytokines, and removing debris or dead cells through phagocytosis. However, aberrant microglia responses, including excessive release of inflammatory cytokines and impaired phagocytosis contribute to neuronal cell death in several neurodegenerative disorders. Microgravity may affect the inflammatory response by microglia and interfere with the cytoskeletal rearrangements necessary for their locomotion or phagocytosis. Thus, understanding microglia (dys)function in neurodegenerative diseases and in LEO may lead to much needed novel targets for therapeutic intervention. None of the current treatments for PD, progressive MS, or other neurodegenerative diseases targets glia cells and neuroinflammation.

PD symptoms are caused by loss of dopamine neurons in the CNS and the standard of care is administration of L-Dopa, the biochemical precursor of dopamine, which enables remaining neurons to release more dopamine; but it does not halt the loss of dopamine neurons. Immuno-modulatory therapies in MS are largely successful in blocking or preventing peripheral immune cell infiltration during the relapsing-remitting phase of MS (RRMS), but primary progressive (PP) and secondary progressive (SP) MS are more challenging to treat. Investigating the glia-induced pathogenic mechanisms in MS, PD, and other neurodegenerative diseases will open up new avenues for more effective treatments.

With further regard the possibility of new treatments, induced pluripotent stem cell (iPSC) technology has revolutionized CNS research allowing human CNS cell types to be studied in culture. Somatic cells, such as peripheral blood mononuclear cells or skin fibroblasts, can be easily reprogrammed to generate iPSCs, which are equivalent to embryonic stem cells in their ability to self-renew and differentiate into any mature cell type. iPSC-derived CNS cells carry the donor's genetic information, enabling unprecedented genotype-phenotype correlation studies to address the genetic complexity underlying neurodegenerative diseases in humans.

Along these lines, three-dimensional human CNS models, often referred to as brain organoids, contain a full complement of neurons and glia, mimicking features of developing brains and capturing cell-cell interactions. Brain organoids have been engineered to carry specific disease-associated mutations and used to model mental disorders such as schizophrenia, neurodevelopmental, and neurodegenerative disorders.

Accordingly, systems and methods that make use of iPSCs organoids with integrated iPSC-derived microglia to model neurodegeneration and neuroinflammation and that allow for the establishment of a culture system that enables long-term survival, including under microgravity conditions, would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes methods and systems for culturing an organoid. In particular, certain embodiments of the presently-disclosed subject matter include methods and systems for culturing an organoid that allow the organoid to be cultured under closed conditions and/or microgravity conditions for an extended period of time.

In some embodiments of the presently-disclosed subject matter, a method of culturing an organoid is provided. In some embodiments, an exemplary method of culturing an organoid includes a first step of combining a neural precursor cell with a first volume of a cortical media or a dopaminergic media to form an organoid. A microglia is then added to the organoid, and the combined organoid and microglia is subsequently added to a cryovial with a second volume of the cortical media or the dopaminergic media, with the second volume of the cortical media or the dopaminergic media including an amount of interleukin (IL)-34 and granulocyte-macrophage colony-stimulating factor (GM-CSF), and an amount of a buffering solution. The cryovial is then sealed and the organoid is cultured for a pre-determined period of time under closed conditions.

In some embodiments of the presently-described methods, the neural precursor cell and/or the microglia is derived from a neural stem cell such as, in certain embodiments, an iPSC.

In some embodiments, the iPSC is obtained from a healthy subject or a subject having a neurodegenerative disease. Such neurodegenerative diseases can, in certain embodiments, be selected from Multiple Sclerosis and Parkinson's disease.

With further regard to the combining of the neural precursor cell with the first volume of a cortical media or a dopaminergic media, in some embodiments, cortical media is utilized and includes an effective amount of cyclic adenosine monophosphate (cAMP), brain-derived neurotrophic factor (BDNF), glial cell-derived neurotrophic factor (GDNF), and neurotrophin (NT)-3. In some embodiments, the cAMP is included at a concentration of about 100 mM, the BDNF is included at a concentration of about 10 ng/ml, the GDNF is included at a concentration of about 10 ng/ml, and the NT-3 is included at a concentration of about 10 ng/ml. In some embodiments that make use of cortical media, the IL-34 is included at a concentration of about 100 ng/ml and the GM-CSF is included at a concentration of about 10 ng/ml.

In other embodiments of the presently-described methods, dopaminergic media is utilized and combined with a neural precursor cell, where the dopaminergic media includes an effective amount of BDNF, GDNF, TGF-β3, ascorbic acid, and cAMP. In some embodiments that make use of the dopaminergic media, the BDNF is included at a concentration of about 20 ng/ml, the GDNF is included at a concentration of about 20 ng/ml, the TGF-β3 is included at a concentration of about 1 ng/ml, the ascorbic acid is included at a concentration of about 200 μM, and the cAMP is included at a concentration of about 500 μM. In some embodiments, the IL-34 included in the dopaminergic media is included at a concentration of about 100 ng/ml and the GM-CSF is included at a concentration of about 10 ng/ml.

In culturing the cells, in some embodiments, culturing the cells for a predetermined period of time comprises culturing the cells for the predetermined period of time under microgravity conditions. In some embodiments, the pre-determined period of time for culturing the cells is at least 28 days. Following the predetermined time period, if desired, the organoid is removed from the cryovial and cultured in a tissue culture vessel. In some embodiments, the organoid comprises a single organoid.

Further provided, in some embodiments of the presently-disclosed subject matter, are systems for culturing an organoid. In some embodiments, a system for culturing an organoid is provided that comprises: an organoid formed from a neural precursor cell; a microglia; an effective amount of a cortical media or a dopaminergic media; a buffering solution; and a cryovial for housing the organoid, the microglia, the cortical media or the dopaminergic media, and the buffering solution under closed conditions.

Similar to the methods described above, in some embodiments of the presently-described systems, the neural precursor cell and/or the microglia is derived from a neural stem cell, such as an iPSC that can be obtained from a healthy subject or a subject having a neurodegenerative disease. In some embodiments of the systems, the neurodegenerative disease is selected from Multiple Sclerosis and Parkinson's disease.

The cortical media included in an exemplary system also includes, in certain embodiments, an effective amount of cAMP, BDNF, GDNF, and NT-3. In some embodiments, the cAMP is included in the cortical media at a concentration of about 100 mM, the BDNF is included at a concentration of about 10 ng/ml, the GDNF is included at a concentration of about 10 ng/ml, and the NT-3 is included at a concentration of about 10 ng/ml.

The dopaminergic media included in an exemplary system then similarly includes, in certain embodiments, an effective amount of BDNF, GDNF, TGF-β3, ascorbic acid, and cAMP. In some embodiments of the dopaminergic media, the BDNF is included at a concentration of about 20 ng/ml, the GDNF is included at a concentration of about 20 ng/ml, the TGF-β3 is included at a concentration of about 1 ng/ml, the ascorbic acid is included at a concentration of about 200 μM, and the cAMP in included at a concentration of about 500 μM.

In some embodiments of the systems, the cortical media or the dopaminergic media comprises an amount of IL-34 and GM-CSF. In some embodiments, the IL-34 is included at a concentration of about 100 ng/ml and the GM-CSF is included at a concentration of about 10 ng/ml.

Further features and advantages of the present invention will become evident to those of ordinary skill in the art after a study of the description, figures, and non-limiting examples in this document.

### BRIEF DESCRIPTION OF THE DRAWINGS

FIG. 1 is a schematic diagram showing an experimental strategy utilized during the course and development of the presently-disclosed subject matter, and depicting hiPSC lines and differentiation protocols.

FIG. 2 is an image of a CubeLab used in accordance with the presently-disclosed subject matter, and showing the CubeLab floating onboard the International Space Station (ISS) while containing human three-dimensional (3D) brain organoids derived from Parkinson's disease (PD) and Primary Progressive Multiple Sclerosis (PPMS) patients.

FIG. 3 includes an image showing the CubeLab held the constant temperature of static culture systems in a 30-day mission onboard the ISS (top) along with images of vials containing organoids at different days and acquired from the CubeLab during the incubation on the ISS (bottom).

FIG. 4 is an image showing the Power Ascent Utility Locker (PAUL) used in accordance with the presently-disclosed subject matter and which provides power and a data interface for monitoring the experiment during launch and on the ISS.

FIGS. 5A-5F include images showing brain organoids cultured in static systems without media exchange are viable after 30 days, including: (FIG. 5A) an image showing the increase in the size of organoids cultured in closed static systems, from the pre-flight stage to the post-flight stage in low-Earth orbit (LEO) and ground; (FIG. 5B) an image showing plated organoids with outgrowth of neural projection and radial glia (arrows) after 30 days culture in low-Earth orbit (LEO) and ground, where the organoids maintained the typical cyto-architecture as shown in the cortical healthy control organoid, and where the neural rosettes are visible and diffused throughout the organoid; (FIGS. 5C-5F) images showing MAP2 and Hoechst staining and tridimensional (3D) rendering displaying the healthy architecture of the neural rosettes (arrows) after 30 days culture in static systems. (HC=healthy control, PPMS=primary progressive multiple sclerosis, PD=Parkinson's disease, LEO=low-Earth orbit).

FIGS. 6A-6I include graphs and plots showing that microgravity alters gene expression in cortical and dopaminergic brain organoids, including: (FIG. 6A) a principal component analysis (PCA) of RNA-seq data showing the clustering of the samples, where PC1 separates dopaminergic vs cortical, and PC2 separates control vs. patient; (FIGS. 6B-6E) plots showing differentially expressed genes (DEGs) analyzed using DESeq2, where the volcano plots were created using the packages ggplot2 and ggrepel, where the log₂ fold change indicates the mean expression for each gene, where genes with significant enrichment (padj<0.05) are shown as red or blue (log₂ fold change >1 or <−1, respectively) across replicates, where the plots shown in FIGS. 6B-6C show the differentially expressed genes (DEGs)—low-Earth orbit (LEO) versus ground—of the iPSC-derived cortical organoids from the healthy control and the iPSC-derived organoids from the multiple sclerosis patients, and where the volcano plots in FIGS. 6D-6E show the DEGs of the iPSC-derived dopaminergic organoids from the healthy control and from the Parkinson's disease patient, and the iPSC-derived organoids (the DEGs were identified using RNA-seq reads quantified from: cortical control ground n=6, cortical control LEO n=5, MS patients ground n=4, MS patients LEO n=4, dopaminergic control ground n=5, dopaminergic control LEO n=5, Parkinson's disease patients ground n=7, Parkinson's disease patients LEO n=6); and (FIGS. 6F-6I) gene enrichment analyses (GSEA) of cortical control, MS patient (FIGS. 6F-6G), dopaminergic control, and PD patient's DEGs (FIGS. 6H-6I) using the org.Hs.eg.db annotation data (pvalueCutoff=0.05, nPermSimple=10000, padj method=Bonferroni, keyType=ENSEMBL).

FIGS. 7A-7B includes graphs and plots showing that microgravity is the cause of the differential gene expression in dopaminergic and cortical organoids, where differentially expressed genes (DEGs) were analyzed using DESeq2 and the volcano plots are created using the packages ggplot2 and ggrepel, where FIG. 7A shows a plot of the differentially expressed genes (DEGs)—low-Earth orbit (LEO) versus ground—of the iPSC-derived 3D organoids (cortical and dopaminergic organoids) from the two healthy controls, and the iPSC-derived organoids from the Multiple Sclerosis and Parkinson's patients (genes with significant enrichment (padj<0.05) are shown as red or blue (log₂ fold change >1 or <−1, respectively) across replicates (LEO samples n=20, ground samples n=22)), and where FIG. 7B shows the gene enrichment analyses (GSEA) of all neurons using the org.Hs.eg.db annotation data (pvalueCutoff=0.05, nPermSimple=10000, padj method=Bonferroni, keyType=ENSEMBL).

FIGS. 8A-8G include graphs and plots showing microgravity affects the secretome of cortical and midbrain organoids cultured in microgravity, where the cortical and midbrain organoids showed a good clustering (FIGS. 8A-8C), where the volcano plot in FIG. 8B for cortical organoids and in FIG. 8D for midbrain shows the differentially secreted proteins (DSPs) (NPX significant are showed in red and non-significant values in turquoise (padj<0.05) (cortical ground=4, cortical LEO=12, dopaminergic ground=4, dopaminergic LEO=11)), where the Venn diagram (FIG. 8G) shows the total protein analyzed emphasizing the DSPs in cortical and dopaminergic samples alone and the DSPs commonly secreted, and where (FIGS. 8E-8F) the GO terms (BP, CP, MP) are shown for cortical and dopaminergic respectively and were performed using shinyGO.

FIGS. 9A-9H are graphs and plots showing differentially expressed genes (DEGs, where the volcano plots of FIGS. 9A-9B show the DEGs—LEO vs ground—of the iPSC-derived cortical control organoids (−MG) and (+MG), where the plots shown in FIGS. 9C-9D show the iPSC-derived MS patient organoids (−MG) and (+MG), where the volcano plots shown in FIGS. 9E-9F show the DEGs of the iPSC-derived DA control organoids (−MG) and (+MG), where the DEGs of the iPSC-derived PD patient organoids are shown in the plots of FIGS. 9G-9H, and where the DEGs are identified using RNA-seq reads quantified with DESeq2. The volcano plots showing the −log(pvalue) and the log₂ fold change, were created using the package ggplot2 and ggrepel. Genes with significant enrichment (padj<0.05) are shown as red or blue (log₂ fold change >1 or <−1, respectively) across replicates (cortical control (−MG) ground n=3, cortical control (+MG) ground n=3, cortical control (−MG) LEO n=2, cortical control (+MG) LEO n=3, MS (−MG) ground n=2, MS (−MG) ground n=2, MS (−MG) LEO n=2, MS (−MG) LEO n=2, DA control (−MG) ground n=2, DA control (+MG) ground n=2, DA control (−MG) LEO n=3, DA control (+MG) LEO n=2, PD (−MG) ground n=4, PD (+MG) ground n=3, PD (−MG) LEO n=4, PD (+MG) LEO n=2).

FIGS. 10A-10D includes graphs and plots, including: (FIG. 10A) a volcano plot showing the DEGs—LEO vs ground—of all (cortical and dopaminergic) iPSC-derived control organoids; (FIG. 10B) a plot showing the DEGs of all (MS and PD) iPSC-derived organoids; and FIGS. 10C-10D plots showing the gene enrichment analysis of the DEG shown in FIGS. 10A-10B, respectively, where the DEGs are identified using RNA-seq reads quantified with DESeq2. The volcano plots showing the −log(pvalue) and the log₂ fold change, were created using the package ggplot2 and ggrepel. Genes with significant enrichment (padj<0.05) are shown as red or blue (log₂ fold change >1 or <−1, respectively) across replicates (control ground n=11, control LEO n=10, patients ground n=11, patients LEO n=10). In FIGS. 10C-10D, the gene enrichment analyses (GSEA) of “all controls” and “all Patients” respectively were report. The GSEA used the org.Hs.eg.db annotation data with pvalueCutoff=0.05, nPermSimple=10000, padj method=Bonferroni, keyType=ENSEMBL.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK© and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is based, at least in part, on the discovery of a system and method that allows for the long-term culture of human iPSC-derived brain organoids (cortical and dopaminergic neural precursor cells) with microglia in closed systems. In particular, in certain embodiments of the presently-disclosed subject matter, brain organoids are maintained in closed cryovials suspended in 1 mL of medium that ensures brain organoid growth and survival without media exchange during long-term culture in microgravity and on Earth. The systems and methods allow for the maintenance of live and metabolically active organoids in culture for at least 28 days in sealed tubes under close conditions or, in other words, without the addition of freshly reconstituted media or repeated equilibration of CO₂ concentration.

In one exemplary implementation for culturing an organoid in accordance with the presently-disclosed subject matter, a neural precursor or progenitor cell is first formed. The phrase “neural precursor cell” and grammatical variations thereof is used herein to refer to the progenitor cells of the central nervous system (CNS) of a subject and which typically give rise the glial and neuronal cell types that populate the CNS, but generally do not generate the non-neural cells that are also present in the CNS, such as immune system cells. In some embodiments, the neural precursor cell used to form an organoid is derived from a neural stem cell, such as, in some embodiments, an iPSC that, as would be recognized by those in the art, is derived from an adult cell but has been reprogrammed back into a pluripotent state (see, e.g., Paull, et al. Nat Methods 12:885-892 (2015), which is incorporated herein by reference in its entirety).

In certain embodiments that make use of an iPSC or other stem cells to form a neural precursor cell, the iPSC or other stem cell is derived or obtained from the cells of a normal healthy subject. As used herein, the term “subject” includes both human and animal subjects such that veterinary uses and applications are within the scope of the presently-disclosed subject matter. The presently-disclosed subject matter thus provides for the culturing of organoids produced or derived from mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the culture of cells from birds, including those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the culture of cells from livestock, including, but not limited to, domesticated swine, ruminants, ungulates, horses (including racehorses), poultry, and the like.

In other embodiments that make use of an iPSC to form a neural precursor cell, the iPSC is derived or obtained from the cells of a subject having a neurodegenerative disease, which is generally characterized by a progressive loss in the structure or function of the neurons in a subject or, in other words, neurodegeneration. Such neurodegenerative diseases include, but are not limited to, disorders including Alzheimer's disease, Parkinson's disease, Huntington's disease, Multiple Sclerosis, Amyotrophic Lateral Sclerosis, Batten disease, and Creutzfeldt-Jakob disease. In some embodiments, the iPSCs or other stem cells used in accordance with the presently-described systems and methods are derived or otherwise obtained from a subject having Multiple Sclerosis or a subject having Parkinson's disease.

Regardless of the particular source of the stem cell, once the stem cells (e.g., the iPSCs) have been obtained, the cells are then differentiated into cortical or dopaminergic neural precursor cells by combining the stem cells with a first volume of a cortical media or a dopaminergic media to form an organoid. Organoids, as would also be recognized by those skilled in the art, are small, self-organized three-dimensional tissue constructs that are typically derived from stem cells and which can be directed to assume a particular cellular identity such that the organoid, at least to a certain degree, mimics its corresponding in vivo organ or tissue. In this regard, the term “cortical media” is used herein to refer to cell culture media capable of being used to differentiate stem cells, such as iPSCs, into a cortical neural precursor cell. Similarly, the term “dopaminergic media” is used herein to refer to cell culture media capable of being used to differentiate stem cells into a dopaminergic neural precursor cell.

The cortical media or dopaminergic media used in accordance with the systems and methods of the presently-disclosed subject matter typically will include an amount of nutrients, growth factors, and the like sufficient to differentiate the neural precursors cells into and maintain the cells as a desired phenotype. As such, the term “effective amount” is used herein to refer to an amount of nutrients, growth factors, and the like that is included in a particular cell culture media and is sufficient to produce and/or maintain a desired phenotype, such as a desired type of neural precursor cell or neural cell or tissue. Actual types and levels of active ingredients in a cell culture media of the present invention can be varied so as to administer an amount of the active compound(s) that is effective to achieve the desired cell type or organoid for a particular system and/or method. Of course, the effective amount in any particular case will depend upon a variety of factors and differentiation into a desired cell type, but the determination and adjustment of an effective amount, as well as evaluation of when and how to make such adjustments, are known to those of ordinary skill in the art. For additional information and guidance regarding the use of methods and cell culture media useful for differentiation of stem cells into cortical and dopaminergic cells, see, e.g., Yao, et al. Cell Stem Cell 20: 120-134 (2017), which is incorporated herein by reference in its entirety. See also, Kriks, et al. Nature 480: 547-551(2011), which is further incorporated herein by reference in its entirety.

With further regard to the cell culture media itself, in some embodiments and implementations, combining the neural precursor cell with the first volume of a cortical media or a dopaminergic media comprises combining the neural precursor cell with cortical media. In some embodiments, the cortical media includes an effective amount of DMEM-F12 media, neurobasal media, cAMP, BDNF, GDNF, and NT-3. In some embodiments, the cAMP is included at a concentration of about 100 mM, the BDNF is included at a concentration of about 10 ng/ml, the GDNF is included at a concentration of about 10 ng/ml, and the NT-3 is included at a concentration of about 10 ng/ml.

In other embodiments and implementations of the presently-disclosed subject matter, combining the neural precursor cell with the first volume of a cortical media or a dopaminergic media comprises combining the neural precursor cell with dopaminergic media. In some embodiments, the dopaminergic media includes an effective amount of neurobasal media, N2 supplemental media, B27 complete, Glutamax, NEAA, Pen/Strep, BDNF, GDNF, TGF-β3, ascorbic acid, and cAMP. In some such embodiments, the BDNF is included at a concentration of about 20 ng/ml, the GDNF is included at a concentration of about 20 ng/ml, the TGF-β3 is included at a concentration of about 1 ng/ml, the ascorbic acid is included at a concentration of about 200 μM, and the cAMP is included at a concentration of about 500 μM.

To assist in the formation of an organoid, following the initial culture of the neural precursor cells with a volume of cortical or dopaminergic media and the resulting formation of an organoid, a microglia, such as a microglia derived from a neural stem cell (e.g., an induced pluripotent stem cell derived (iPSC)), is then added to the organoid. As would be recognized by those skilled in art, microglia function as macrophages in the central immune system and play important roles in the development and maintenance of the central nervous system. It is further appreciated that stem cells can be differentiated to form microglia (see Douvaras, et al. Stem Cell Rep. June 6; 8(6): 1516-1524 (2017), which is also incorporated herein by reference) and, in this regard and without wishing to be bound by any particular theory or mechanism, it was believed that the use of matched microglial cells that were differentiated in parallel from the same iPSCs and then added to the organoids for integration were useful for mimicking the migration of microglial progenitors to the developing nervous system during differentiation and thereby promoting the development of the organoid.

In some embodiments, to encourage microglia maturation after incorporation in the organoids, the combined organoid and iPSC-derived microglia are subsequently added to a cryovial with a second volume of the cortical media or the dopaminergic media that then also includes an amount of IL-34 and GM-CSF, as well as an amount of a buffering solution. In some embodiments and implementations of the methods and systems of the presently-disclosed subject matter that make use of cortical media, the media further includes IL-34 at a concentration of about 100 ng/ml and GM-CSF at a concentration of about 10 ng/ml. In some embodiments and implementations that make use of dopaminergic media, the IL-34 is included at a saturating concentration of at least about 100 ng/ml and the GM-CSF is included at a concentration of about 10 ng/ml.

With regard to the cryovials used in accordance with the presently-described systems and methods, although the term “cryovial” is often used in relation to containers capable of withstanding low temperatures (e.g., −196° C.), the use of the term “cryovial” herein is not limited to such containers but is further inclusive of any sufficiently durable container capable of being sealed tightly and utilized for the long term storage of cell and tissues. In some embodiments, the cryovials capable of use in accordance with the presently-disclosed subject matter can be obtained from commercially-available sources such as, for example, the NUNC™ Coded Cryobank Vial from Thermo Fisher Scientific.

As indicated above, in addition to the inclusion of IL-34 and GM-CSF, a buffering agent can also be added to the cell culture media included in the cryovial to maintain the pH of the media during the culturing of the organoids under closed conditions. In some embodiments, the buffering solution comprises HEPES (4-(2-hydroxyethyl)-1-piperazineethanesulfonic acid), as it was believed that the use of this zwitterionic sulfonic acid buffering agent could maintain physiological pH despite potential changes in carbon dioxide concentration during the course of the culturing of the organoid for a period of time under closed conditions in the cryovial. In some embodiments, the HEPES is included in the cell culture media at a concentration of 10-15 mM. In some embodiments, and without wishing to be bound by any particular theory or mechanism, it is believed that the addition of the HEPES and the relatively small size of the organoid relative to the larger volume of media provides an environment capable of supporting the organoid for a month or more. More specifically, it is contemplated that, in certain embodiments, seeding the organoids with a limited number of cells (e.g., 100K) allows the organoids to grow a limited amount within the one month time frame as the neural precursors proliferate. The organoid then remains relatively small (about 0.5 mm or about less than 0.75 mm) within the volume of media in the cryovials (1-2 ml) such that the ratio of the media volume to the size and/or number of cells assists in the maintenance of the survival of the organoid under the closed conditions.

Upon placing the combined organoid and microglia in the cryovial with the second volume of media including the buffering solution, the cryovial is then sealed, such as by capping the cryovial and sealing it with parafilm, to create a closed system in which the cryovial is completely sealed off from its external environment. The organoid with the microglia and culture media is then cultured for a pre-determined period of time under closed conditions whereby the cryovial and cultured cells included in the cryovial are not re-opened during the culture period. In some implementations, only a single organoid is included per cryovial. In some implementations, the culture period is at least 28 days. In some implementations, the culture period takes part, in total or in part, under microgravity conditions.

Subsequent to the initial culturing, in some implementations, the methods described herein further include the steps of removing the organoid from the cryovial after the predetermined time period, and culturing the organoid in another tissue culture vessel as it has been surprisingly determined that the presently-described systems and methods allow for the culturing of organoids under closed and/or microgravity conditions for an extended period of time and in a manner that maintains the viability of the cultured cells/organoids. In some embodiments, the culturing of the cells following the closed conditions is accomplished using the above-described media with standard cell culture gas conditions (e.g., 5% CO₂, 20% O₂). In some embodiments, the additional culturing is further performed under conditions sufficient to allow the cells to attach to cell culture surfaces coated with extracellular matrix (ECM) proteins such as laminin and/or fibronectin to promote outgrowth of cells and nerve projections onto the surfaces under standard culture conditions as would be appreciated by those skilled in the art.

Still further provided, in some embodiments of the presently-disclosed subject matter, are systems for culturing an organoid. In some embodiments, a system for culturing an organoid is provided that comprises: an organoid derived formed from a neural precursor cell; an induced pluripotent stem cell derived (iPSC)-derived microglia; an effective amount of a cortical media or a dopaminergic media; a buffering solution; and a cryovial for housing the organoid, the microglia, the cortical media or the dopaminergic media, and the buffering solution under closed conditions.

Also provided by the presently-disclosed subject matter are cell culture media and kits. In some embodiments, cell culture media is provided that includes the components and various combinations thereof described herein above for use with the presently-described systems and methods. In some embodiments, a kit for culturing an organoid is provided that includes one or more of the components of the systems of the presently-disclosed subject matter along with instructions for using the kit. In some embodiments, the instructions for using the kit include instructions for carrying out methods for culturing an organoid in accordance with the presently-disclosed subject matter.

By making use of the systems and methods described herein, it has been discovered that the presently-disclosed subject matter allows the survival of cortical and dopaminergic brain organoids in closed long-term culture systems for space mission(s) studies onboard of the international space station (ISS) without the intervention of the astronauts. Further, the methods and systems allow for the culture in microgravity of human brain organoids as a disease model for neurodegenerative disorders—such as multiple sclerosis (MS) and Parkinson's disease (PD). The methods and systems also provide a long-term culture system for biological research during a lunar mission, and the methods and systems will further allow for long-term culture onboard of the lunar station—Gateway—orbiting the Moon in the near future.

Furthermore, it is believed that the methods and systems are important for studies performed on deep space travel such as to Mars, and to understand the effect of differential gravity and cosmic radiation on human brain. The data collected from the studies performed using the culture system described herein will speed-up the human mission to Mars and will make lunar missions safer, contributing to the development of countermeasures for deep space missions. The methods and systems will also improve the culture of brain organoids on Earth, making the organoid maintenance more time-efficient for scientists on Earth.

Lastly, while certain embodiments of the presently-disclosed subject matter specifically make reference to closed systems including brain organoids, it is appreciated that the systems and methods described herein are equally applicable to organoids made from iPSCs or hESCs differentiated into other cell types, such as liver, heart, and skeletal muscle. Any cell type that can be derived from iPSCs may be cultured in accordance with the presently-disclosed subject matter.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, TRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. The Examples may include compilations of data that are representative of data gathered at various times during the course of development and experimentation related to the present invention.

### EXAMPLES

Cultured induced pluripotent stem cells derived from people with neurodegenerative disease can be used to produce human brain organoid models and it was believed that such models could be useful in addressing an urgent unmet need for understanding the mechanisms of neurodegenerative disease, and for studying the effects of microgravity on central nervous system (CNS) function in space travelers. Exposure to microgravity in low-Earth orbit (LEO) has been shown to impact the cardiovascular, musculoskeletal, and immune systems of the human body. Evidence from astronaut data and mouse models suggests that microgravity also causes intracranial fluid shifts and alters white and gray matter. Leveraging induced Pluripotent Stem Cell (iPSC) technology to generate CNS cells, experiments were thus undertaken to develop human models integrating iPSC-derived microglia into three-dimensional (3D) neural organoids derived from the cells of patients with primary progressive multiple sclerosis (PPMS), Parkinson's disease (PD), and their aged-matched non-symptomatic controls. These human models of PD and MS were maintained on the International Space Station (ISS) for 30-days. As described in further detail below, post-flight samples were evaluated using transcriptome and secretome analysis to assess microgravity-induced changes in neuroinflammation pathways. Bulk RNA sequencing analysis showed differentially expressed genes in LEO compared to ground control samples. Gene Set Enrichment Analysis (GSEA) of spaceflight samples also indicated dysregulation of cell division, DNA repair and packaging, and post-translational modifications of proteins. These experiments allowed for the study of these human analog models of PD and MS in LEO to understand the neuroinflammation pathways involved in neurodegenerative disease and identify therapeutic targets.

### Methods

Preparation of Neural Stem Cells (NSCs). Four different lines of iPSCs derived from different healthy control (HC) individuals, a primary progressive multiple sclerosis (PPMS) patient, and a Parkinson's disease (PD) patient (see Table 1) were differentiated into cortical and dopaminergic neural precursors in standard culture dishes. A previously-published protocol for culture dishes was followed for the cortical differentiation methods (see Yao Z, Mich J K, Ku S, et al. A Single-Cell Roadmap of Lineage Bifurcation in Human ESC Models of Embryonic Brain Development. Cell Stem Cell 20:120-134 (2017)) and for the dopaminergic differentiation (see Kriks S, Shim J-W, Piao J, et al. Dopamine neurons derived from human ES cells efficiently engraft in animal models of Parkinson's disease. Nature 480: 547-551 (2011)). Briefly, the MS line and its matching control were reprogrammed from dermal fibroblasts with the integration-free, virus-free mRNA/miRNA method, and the PD line and matching control were reprogrammed from dermal fibroblasts using non-integrating Sendai virus). At day 25 of the differentiation, for both cortical and dopaminergic precursors, the precursors were dissociated and cryopreserved in liquid nitrogen, creating a stock of day 25 (d25) of neural precursors (NPCs).

TABLE 1
Cell Lines.

| | | | | | | Somatic | Reprogramming |
|---|---|---|---|---|---|---|---|
| | Species | Cell Line ID | Type/Mutation | Sex | Ethnicity | Cell Line | Method |
| Subject I | Human | 051121-01- | No Known | Female | Caucasian | Biopsy- | mRNA |
| | | MR-017 | Diseases/CLN3 | | | derived | |
| | | | het | | | fibroblast | |
| Subject II | Human | AK003-01-MR- | Primary | Female | Caucasian | Biopsy- | mRNA |
| | | 008 | Progressive | | | derived | |
| | | | Multiple | | | fibroblast | |
| | | | Sclerosis | | | | |
| Subject III | Human | HDF410iPS504 | No Known | Male | Caucasian | Biopsy- | Sendai |
| | | | Diseases | | | derived | |
| | | | | | | fibroblast | |
| Subject IV | Human | UEC741iPS517 | Sporadic | Male | Vietnamese | Epithelial | Sendai |
| | | | Parkinson's | | | cells | |
| | | | Disease | | | from | |
| | | | | | | urine | |
| | | | | | | sample | |

Preparation of IPSC-derived microglia. For the microglia differentiation a previously-published protocol for culture dishes was also followed (see Douvaras P, Sun B, Wang M, et al (2017) Directed Differentiation of Human Pluripotent Stem Cells to Microglia. Stem Cell Rep.).

Preparation of brain organoids. The cryopreserved day 25 (d25) NPCs were thawed, washed in 5 mL DMEM/F12, and harvested by centrifugation at 1200 rpm, 5 min. The cell pellets were re-suspended in 1 mL of cortical medium (see Table 2) (for cortical NPCs) and 1 mL of dopaminergic medium (see Table 2) (for dopaminergic NPCs). After counting the cortical and dopaminergic NPCs, they were plated at a density of 1×10\hat{ }5 cells/well onto ultra-low attachment (ULA) 96-well V bottom plates (PrimeSurface® 3D Culture Spheroid Plates: Ultra-low Attachment (ULA) Plates, Sbio MS-9096VZ) and incubated overnight. at 37° C., 5% CO₂. After 24 hours incubation, the organoids were formed, the spent media was replaced and the organoids were incubated for a further 24 hours. At that stage, in preparation for space launch, iPSC-derived microglia was added to the brain organoids. One media exchange was also performed using the cortical and dopaminergic media with the addition of IL-34 and GM-CSF to encourage microglia maturation after incorporation into the organoids (see Table 2). Those were then incubated for a further 24 hours before shipping them to the International Space Station U.S. National Laboratory for payload preparation and launch. Culture media for cryovials was buffered by HEPES for culture without 5% CO₂. DMEM-F12 and Neurobasal media were purchased from ThermoFisher (Cat. Nos. #11330 and 21103049). N2 supplement (cat. no. 17502048), B27 (cat. no. 17504044), Glutamax (cat. no. 35050061), NEAA (cat. no. 11140050), Pen/Strep (cat. no. 15070063) were each purchased from Gibco/ThermoFisher. BDNF (cat. no. 248-BD/CF), GDNF (cat. no. 212-GD-050), NT3 (cat. no. 267-N3-MTO) and TGF-β3 (cat. no. 243-B3-010) were from R&D Systems. Ascorbic acid (cat. no. A4403-100MG), and cAMP (cat. no. D0627-1G) were purchased from Sigma Aldrich.

TABLE 2
Cortical and Dopaminergic Media.

| Cortical Medium | | | |
|---|---|---|---|
| | [Stock] | [Final] | Volume |

| | | |
|---|---|---|
| DMEM/F12 | 30 | mL |
| Neurobasal | 30 | mL |

| | | | | | | |
|---|---|---|---|---|---|---|
| cAMP | 200 | mM | 100 | mM | 30 | uL |
| BDNF | 40 | μg/mL | 10 | ng/mL | 15 | μL |
| GDNF | 20 | μg/mL | 10 | ng/mL | 30 | μL |
| NT-3 | 100 | μg/mL | 10 | ng/mL | 6 | μL |
| IL-34 | 100 | μg/mL | 100 | ng/mL | 60 | μL |
| GM-CSF | 100 | μg/mL | 10 | ng/mL | 6 | μL |

| Dopaminergic Medium | | | |
|---|---|---|---|
| | [Stock] | [Final] | Volume |

| | | | | |
|---|---|---|---|---|
| Neurobasal | | | 94 | mL |
| N2 | 10X | 1X | 1 | mL |
| B27 complete | | | 2 | mL |
| Glutamax | | | 1 | mL |
| NEAA | | | 1 | mL |
| Pen/Strep | | | 1 | mL |

| | | | | | | |
|---|---|---|---|---|---|---|
| BDNF | 40 | μg/mL | 20 | ng/mL | 50 | μL |
| GDNF | 40 | μg/mL | 20 | ng/mL | 50 | μL |
| TGF-β3 | 10 | μg/mL | 1 | ng/mL | 10 | μL |
| Ascorbic Acid | 200 | mM | 200 | μM | 100 | μL |
| cAMP | 500 | mM | 500 | μM | 100 | μL |
| IL-34 | 100 | μg/mL | 100 | ng/mL | 100 | μL |
| GM-CSF | 100 | μg/mL | 10 | ng/mL | 10 | μL |

Payload preparation, launch, and splashdown. Individual brain organoids were each transferred into a cryovial (NUNC™ Coded Cryobank Vial, Thermo Fisher Scientific #374088), containing 1 mL of cortical or dopaminergic medium with the addition of IL-34 (100 ng/mL) and GM-CSF (10 ng/mL). The cryovials were then closed and sealed with parafilm and loaded into a CubeLab, a miniaturized incubator that maintains a stable temperature of 37° C. The CubeLab was then loaded into the rocket and after two days, launched into low-Earth orbit onboard the SpaceX 19 Commercial Resupply Services mission to the ISS for a 30-day stay in micro-gravity. Under similar conditions, ground control samples were kept at the Kennedy Space Center. The Dragon capsule carrying the organoids back to Earth splashed down in the Pacific ocean, and the CubeLab with the organoids was retrieved and opened six days later.

RNA extraction and sequencing. 22 organoids cultured in LEO and 22 from ground control were snap-frozen and stored at −80° C. RNA was extracted using the Qiagen Rneasy Micro kit (QIAGEN #74004). To maximize the yield, the RNA was eluted into 12 μL of ultrapure DI water. The RNA was sequenced using an ultra-low input preparation of samples with a minimum of 10 ng of RNA and the Illumina NovaSeq Platform for high-throughput sequencing.

Analysis of secreted proteins using Proximity Extension Assay (PE 4). Culture medium from each vial (90 vials) was removed and frozen at −20° C. Proteormic analysis was performed by Olink (Olink Proteormics AB, Uppsala, Sweden) using the Olink@ Explore 1536 Olink protocol technology based on the Proximity Extension Assay (PEA). The assay can detect up to 1536 proteins in 90 samples using pairs of oligonucleotide-barcoded antibodies. The raw output data is converted into “Normalized Protein eXpression” (NPX), Olink's unit of relative abundance. All assay validation data (detection limits, intra- and inter-assay precision data, predefined values, etc.) are available on the manufacturer's website. The secretome data were analyzed using the Olink® Insights Stat application and shinyGO for the GO term analysis.

Differential gene expression and Gene Set Enrichment Analysis (GSEA). The RNA sequencing data were analyzed using the R packages DESeq2, and Gene Set Enrichment Analysis (GSEA) analyzed using clusterProfiler 4.0, Pathview, enrichplot, and ggplot2, and shinyGO for the GO term analysis.

Staining, image acquisition andprocessing. Organoids were fixed with a 4% paraformaldehyde (PFA) solution in PBS for 30 minutes. The organoids were processed following a previously published protocol for whole mounting staining and stained using an antibody to MAP2 (Ab5392, dilution 1:1000) to detect neurites and Hoechst dye for labeling nuclei. The labeled organoids were imaged using a ZEISS confocal microscope (LSM780), and images were processed and edited using Imaris Image Analysis Software.

Flight Hardware. RADTriage50 (JP Laboratories, Inc.) passive dosimeters were included in the flight hardware both on station and on ground. RADTriage50 devices from both LEO and ground units were assessed in post-flight evaluation.

Radiation Environmental Monitoring (REM) methods on ISS. Radiation detectors used on the ISS include Radiation Environment Monitoring (REM) and Hybrid Electronic Radiation Assessor (HERA) devices. Both REM and HERA were operational on the ISS during the experiments. Station monitoring logs adjacent to the payload storage location on the ISS were provided for post-flight evaluation as a secondary method for measurement of radiation exposure during the mission.

### Results

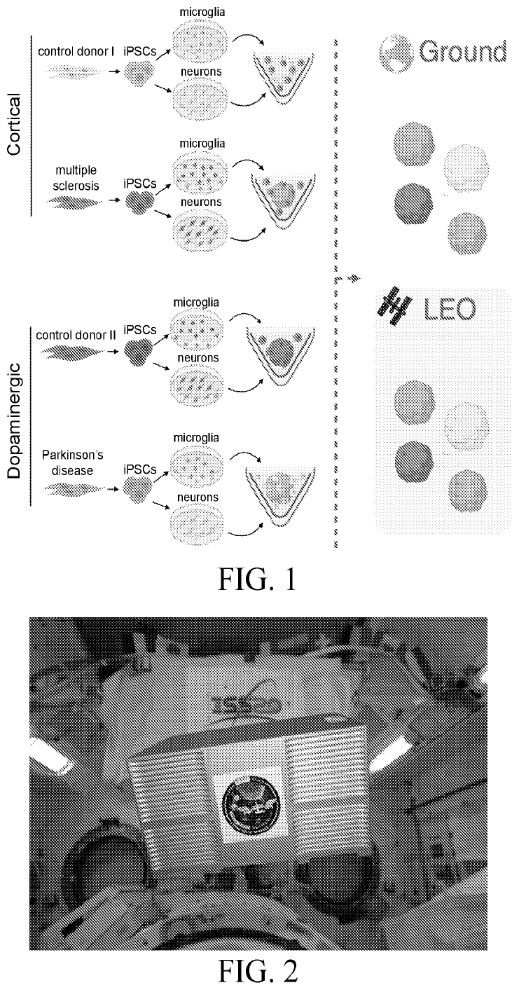

Establishment of Experimental Strategy. Two human brain organoid models were established to investigate the effects of microgravity on brain cells, representing the cortex for MS and the midbrain dopamine system for PD. In initial experiments, two iPSC lines were used per organoid system, generated by reprogramming adult skin fibroblasts or primary urinary epithelial cells obtained from people with PPMS or PD and age and sex-matched healthy donors (see FIG. 1). iPSC-derived microglial progenitors were then generated and incorporated into 3D cultures of dopaminergic organoids for the PD lines or cortical organoids for the MS lines.

Neuronal progenitors were seeded into U-bottom wells to enable aggregation into organoids, and matched microglia progenitors, differentiated in parallel from the same iPSCs, were added to the organoids for integration. This process was intended to mimic the migration of microglial progenitors to the developing CNS during embryonic development. The 3D organoids cultures were shipped to the Space Station Processing Facility (SSPF) at Kennedy Space Center (KSC), where they were transferred to individual tubes in 1 mL of culture medium, sealed, and loaded onto flight hardware for parallel culture on the ground at KSC and in LEO on the ISS.

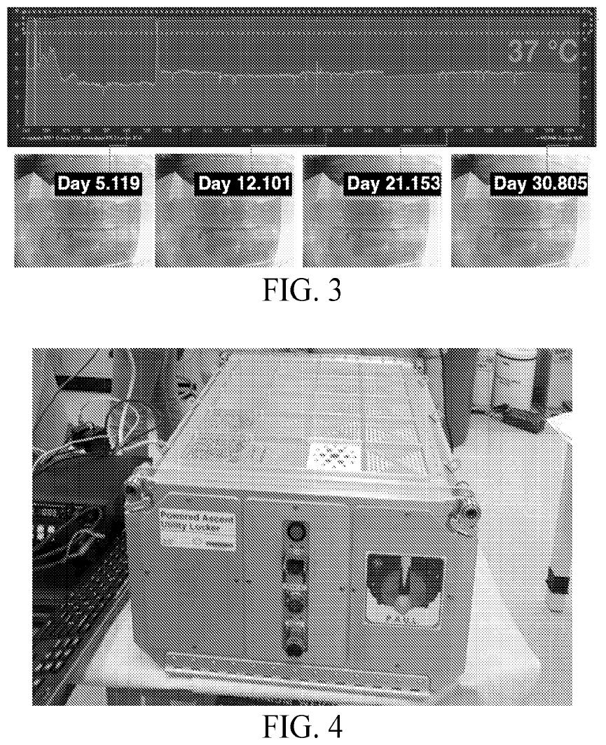

Growth and maintenance of iPSC-derived brain organoids required a sterile environment, a stable temperature of 37° C., and cell type-specific media. To facilitate equivalent cell culture studies in LEO, the CubeLab system (Space Tango, Lexington, KY), a miniaturized incubator that maintained a constant temperature of 37° C. (see FIGS. 2 and 3) and provided imaging capability onboard the ISS during static suspension culture for 30 days, was utilized.

To facilitate the culture of 3D organoids in LEO, operational limitations that might confound the analysis in microgravity were also considered. For example, the timeline from hardware handover to installation on the ISS can vary between 2.5 to 5 days, depending on launch scrubs, launch trajectory of the spacecraft, and station schedules post-docking of the vehicle. These variables could impact biological experiments if they were not maintained under standard conditions. The Powered Ascent Utility Locker (PAUL) facility (Space Tango) was designed and utilized to limit the adverse effects of these unexpected events during launch and transit to the ISS. The CubeLab was installed within the PAUL (see FIG. 4) and was connected directly to the SpaceX Dragon capsule, and provided power to maintain temperature and a data interface for monitoring the experiment until it was installed on the ISS. This capability allowed for continual maintenance of the environmental parameters in the CubeLab's incubator during the experiment's pre-launch, launch, and transit phases to the ISS. On the ISS, the CubeLab was installed within a reconfigurable experiment ecosystems facility for microgravity research aboard the ISS. That facility and the PAUL required a single EXPRESS rack locker volume and provided mechanical, electrical, and network interfacing that allowed monitoring of experimental progress.

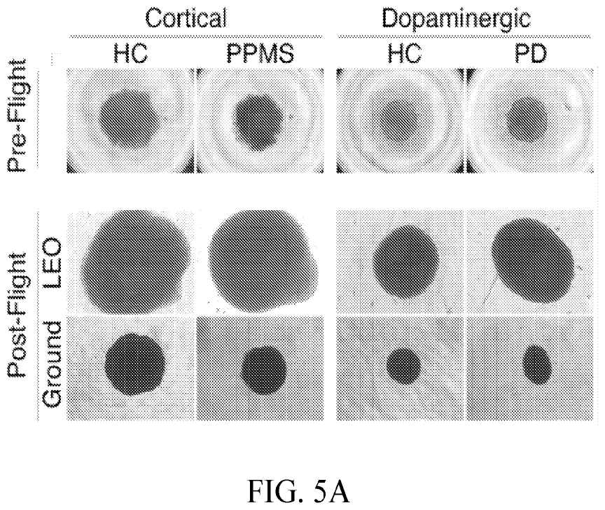

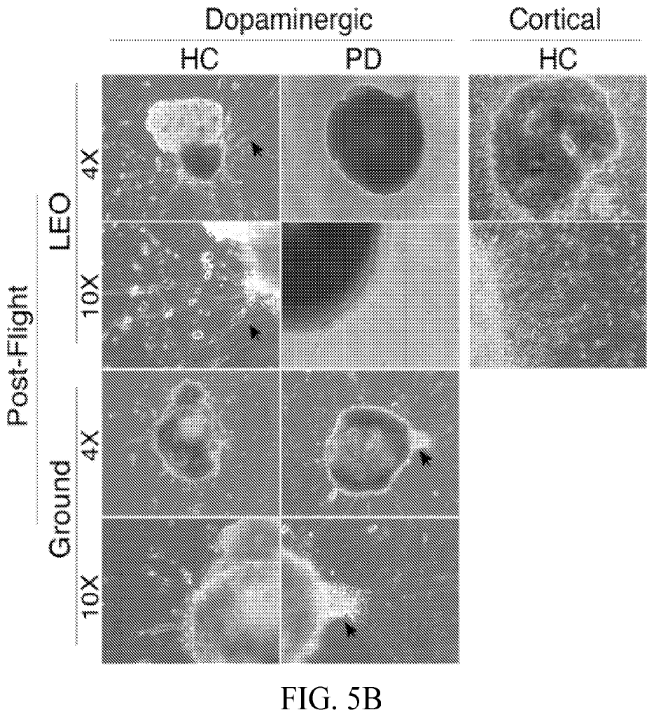

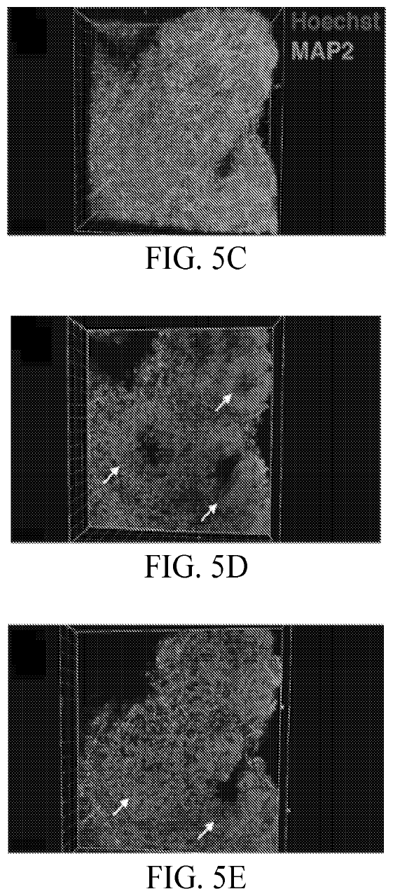

Organoids survive for one month in static culture. Organoids were cultured for approximately a month in one ml of medium in cryovials without medium changes. The organoids increased in size both in LEO and on ground (FIGS. 5A-5B). After return to Earth, a small sample of the organoids were assessed for viability by plating onto laminin-coated dishes in fresh medium. Organoids attached to the plate showed robust process outgrowth, indicating that they thrived during the month-long culture in microgravity. The cortical organoids formed neural rosettes typical of this cell type (FIGS. 5B-5F).

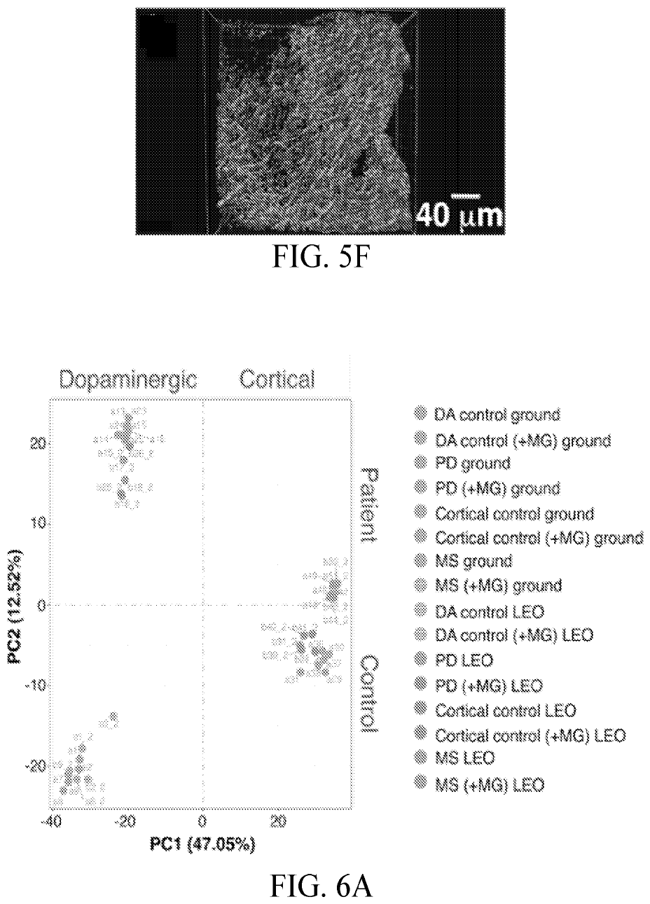

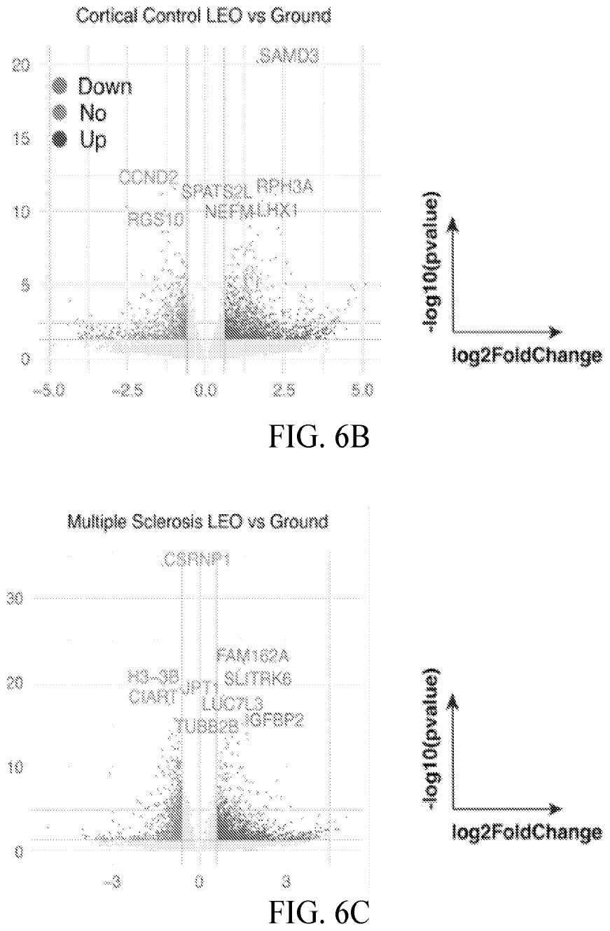

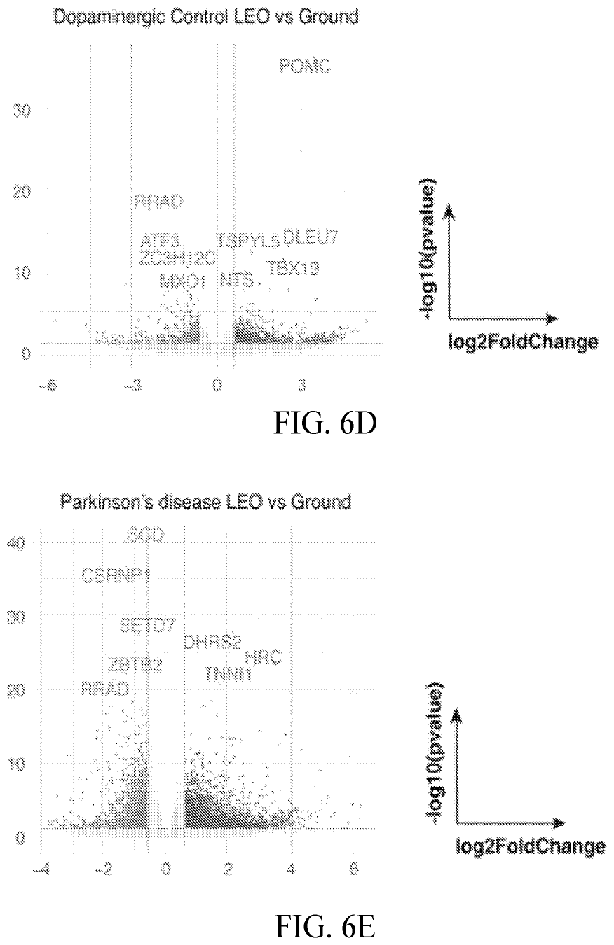

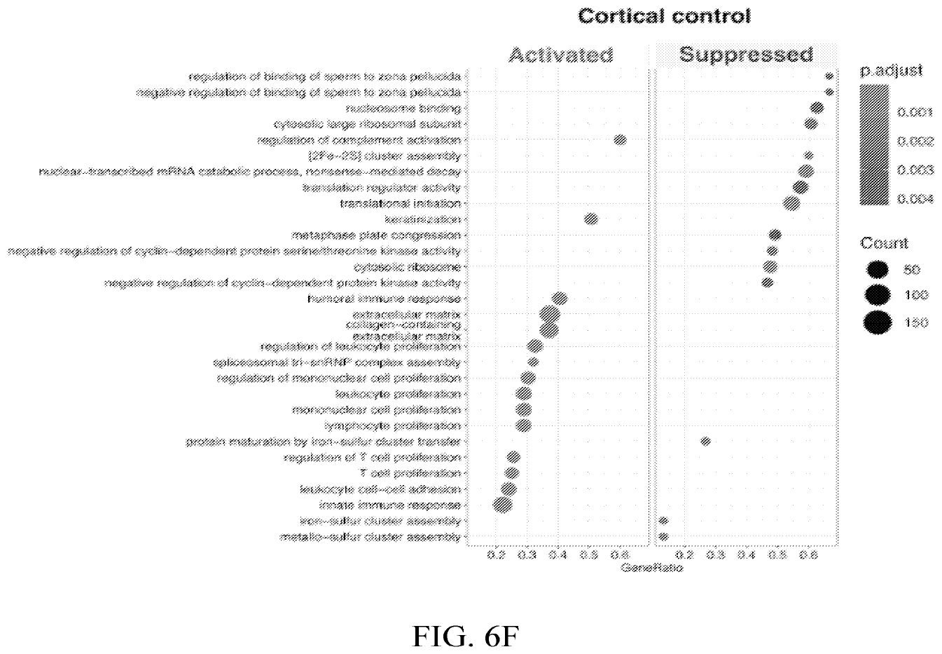

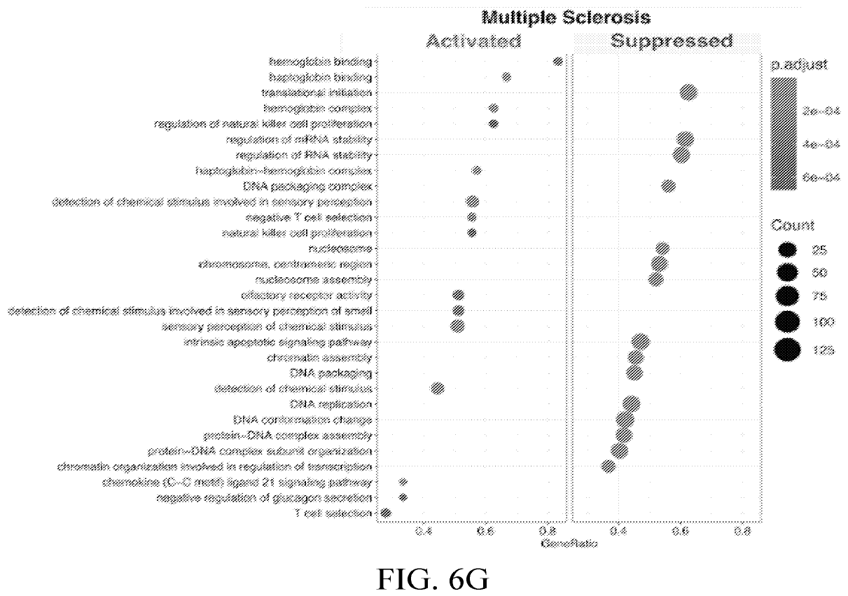

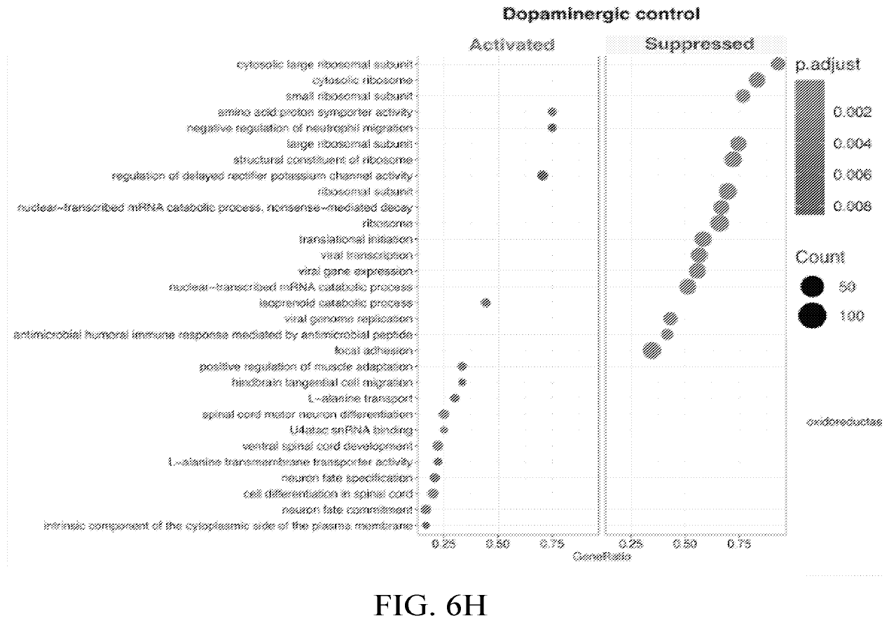

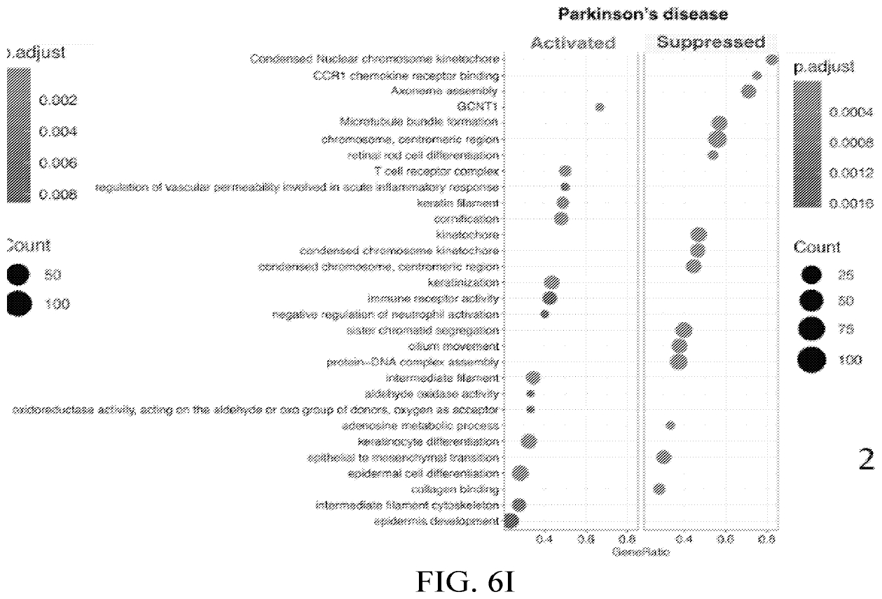

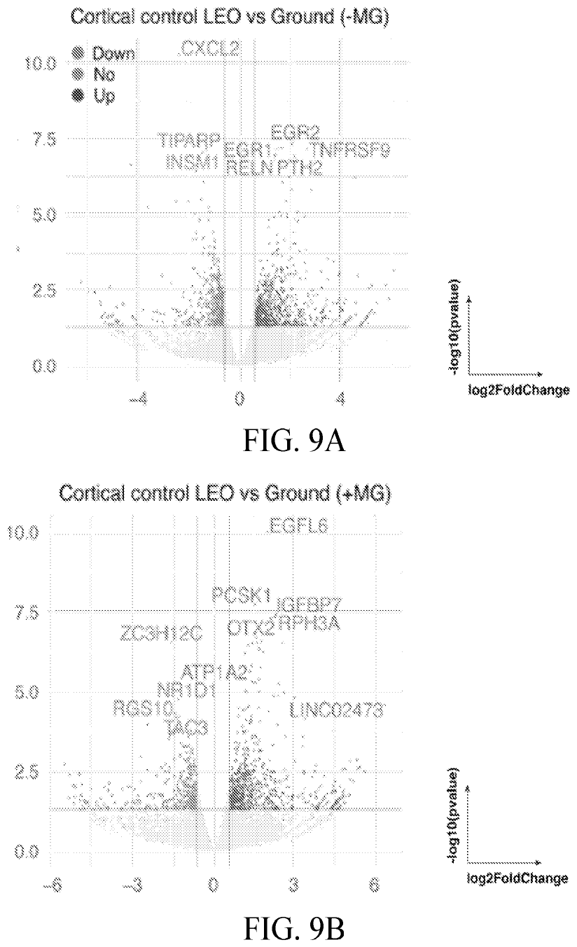

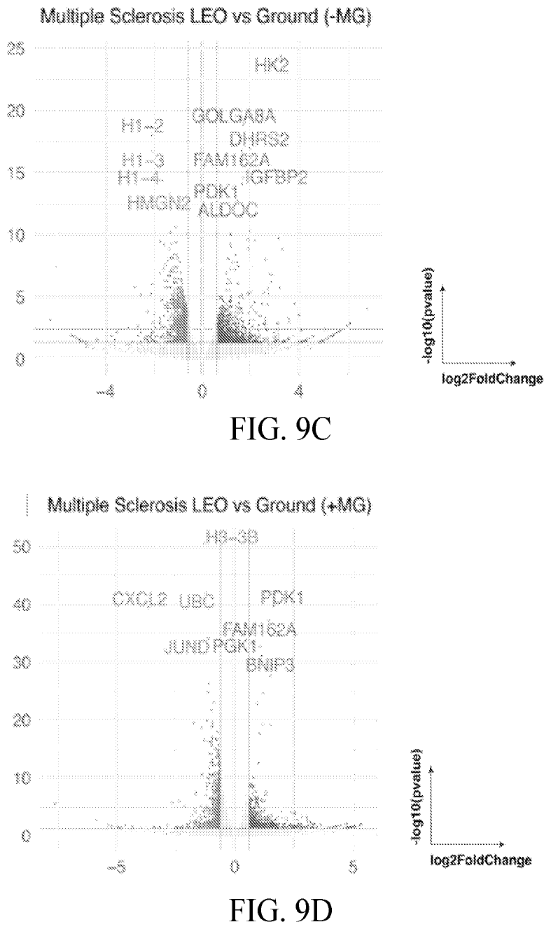

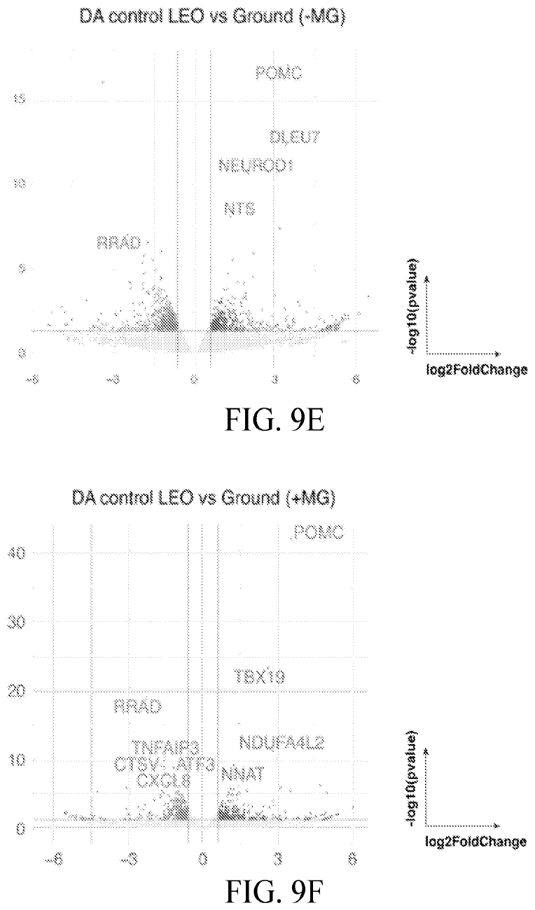

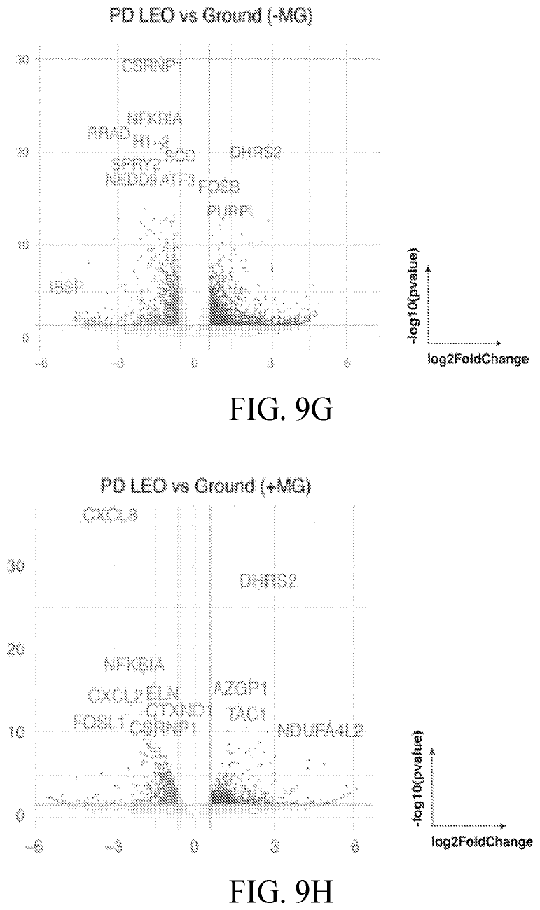

Microgravity affects gene expression profiles of organoids. RNA sequencing was performed on replicate samples (n=2-4 per condition) and gene expression data was analyzed using a DESeq2 package. Principal component analysis of all organoids (FIG. 6A) showed a clear distinction between the dopaminergic and cortical organoids. To determine the effects of LEO associated with each of the organoid types, differences in ground compared to LEO were examined in separate analyses for each of the organoid types (cortical or dopaminergic) for disease-associated cells (PD or MS) and matched controls (FIGS. 6B-6E). DESEq2 identified differentially expressed genes that had a padj<0.05 and log₂ fold change >1 and <−1. For the cortical organoids, 552 LEO vs ground DEGs were found for the non-MS control, and 2,828 DEGs were found for the LEO vs ground comparison of primary progressive multiple sclerosis. There were 160 LEO vs ground DEGs for the non-PD control, and 4,883 for the Parkinson's disease organoids. Organoids with and without microglia were also analyzed (FIGS. 9A-9E). To gain insight about the biological processes affected by microgravity Gene Set Enrichment Analysis (GSEA) was performed on the ground vs LEO DEGs. FIGS. 6F-6I lists the highest ranking differentially expressed gene sets, which include associations with DNA repair, mitotic checkpoint, and maturation in LEO.

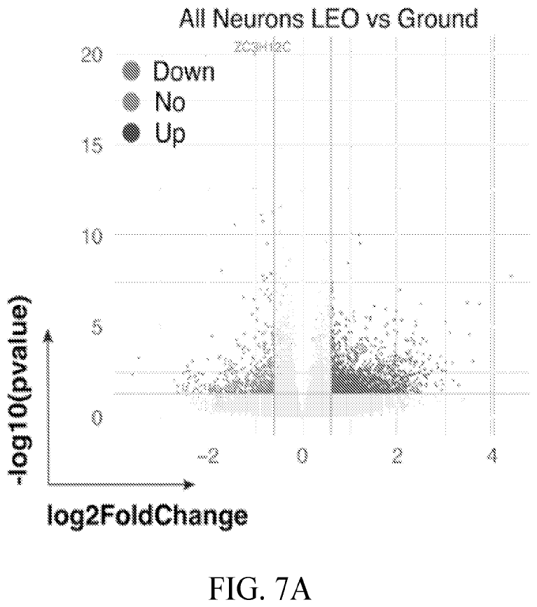

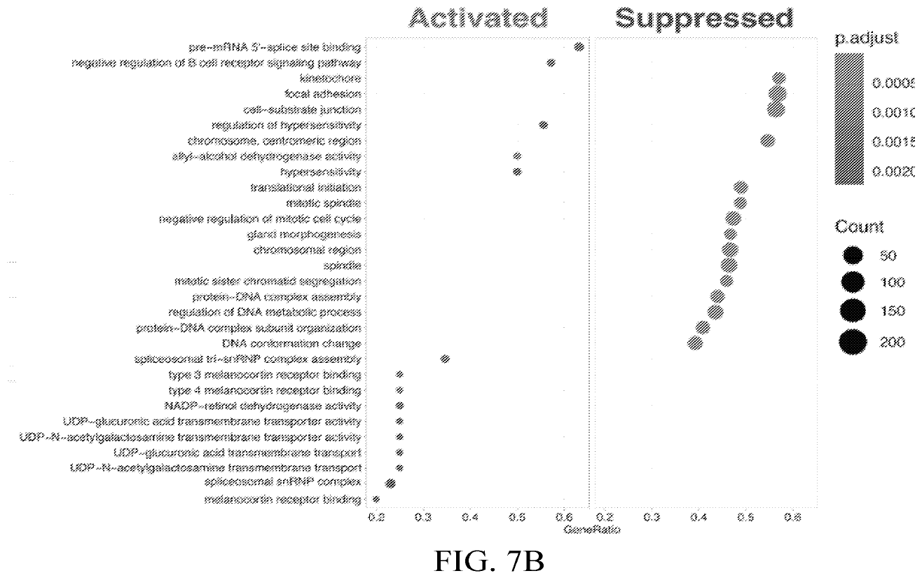

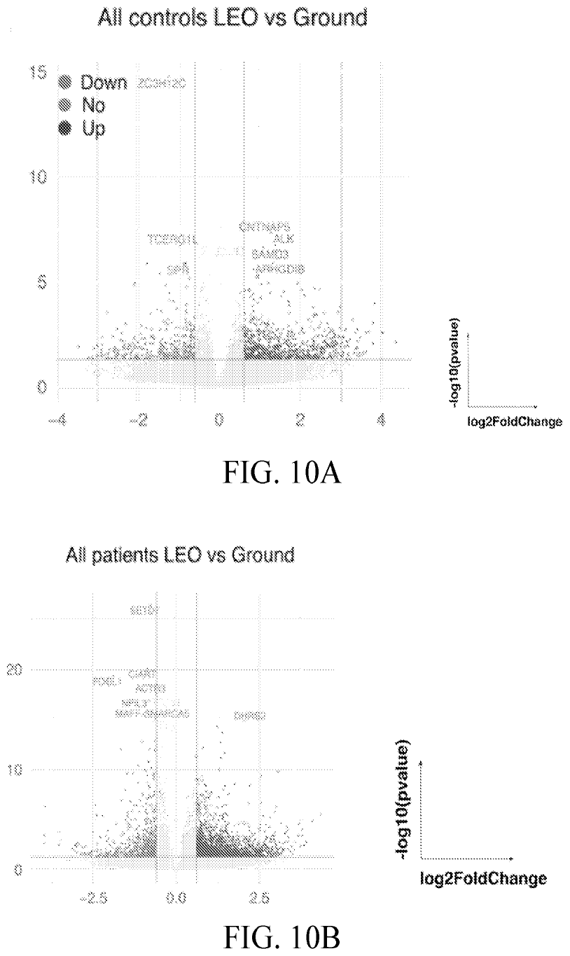

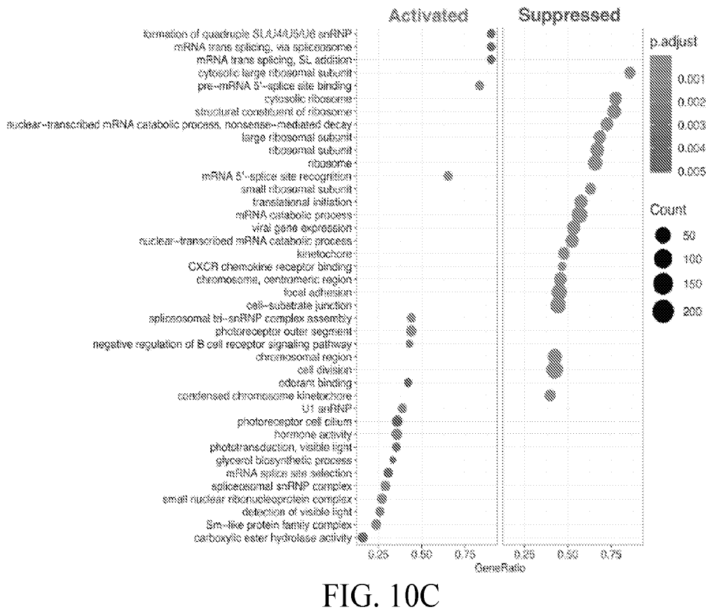

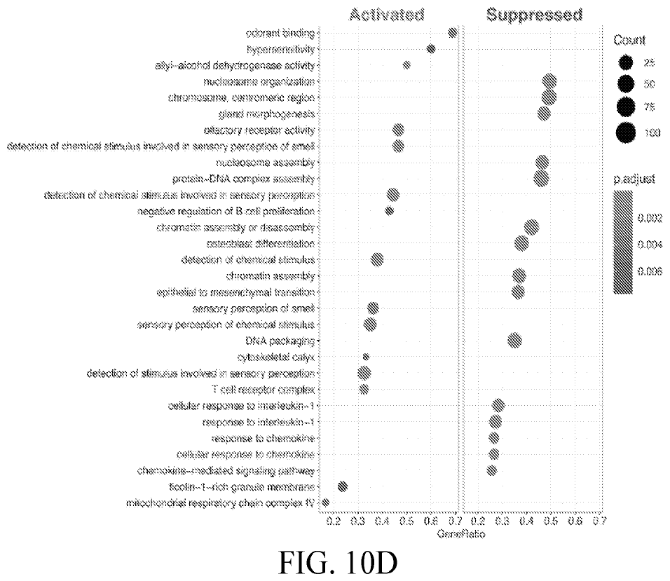

Responses to microgravity were not dependent on person-to-person variability but do differ between organoid types. Because of genomic differences among individuals, the differences in gene expression were examined with emphasis on whether the expression was different depending on the donor. To look for person-to-person variability, the totality of the four individuals was considered rather than subtyping the organoids according to disease type or neuronal type (FIGS. 7A-7B). In addition, the DEGs of all the controls (cortical and dopaminergic) and all the patients (MS and PD) were analyzed separately (FIGS. 10A-10D). DESeq2 identified genes that were differentially expressed which had a padj<0.05 and log_{z} fold change >1 and <−1. 917 DEGs were reported for the “all neurons,” 66 for the “all controls,” and 1834 for the “all patients”.

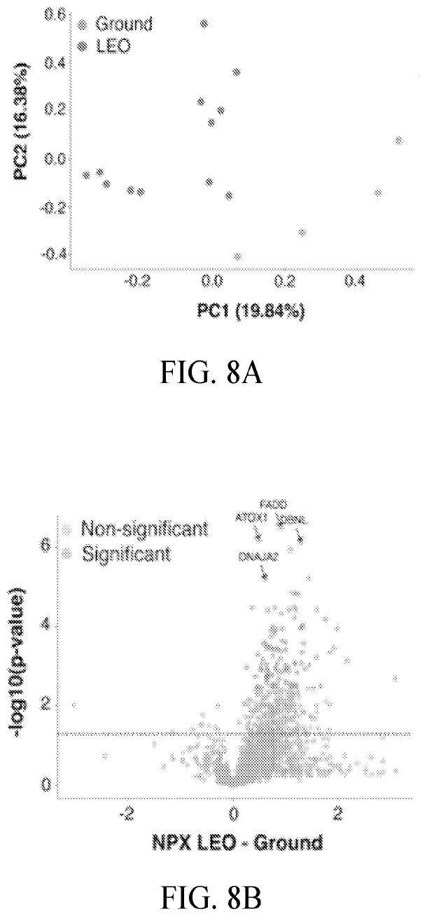

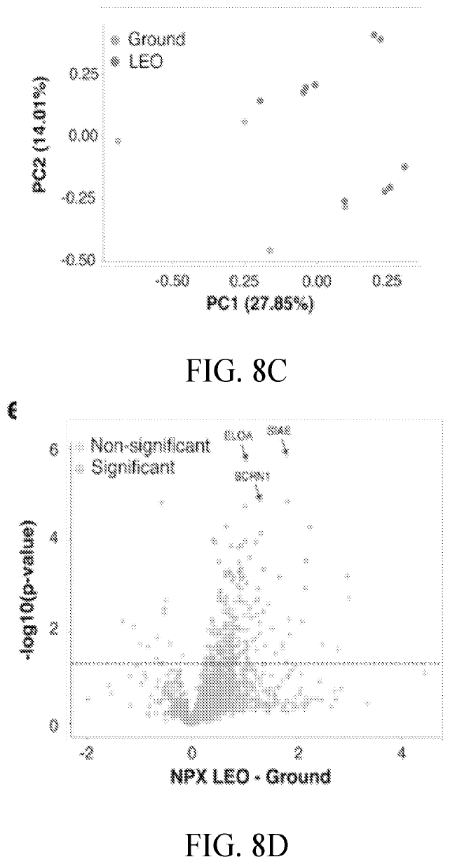

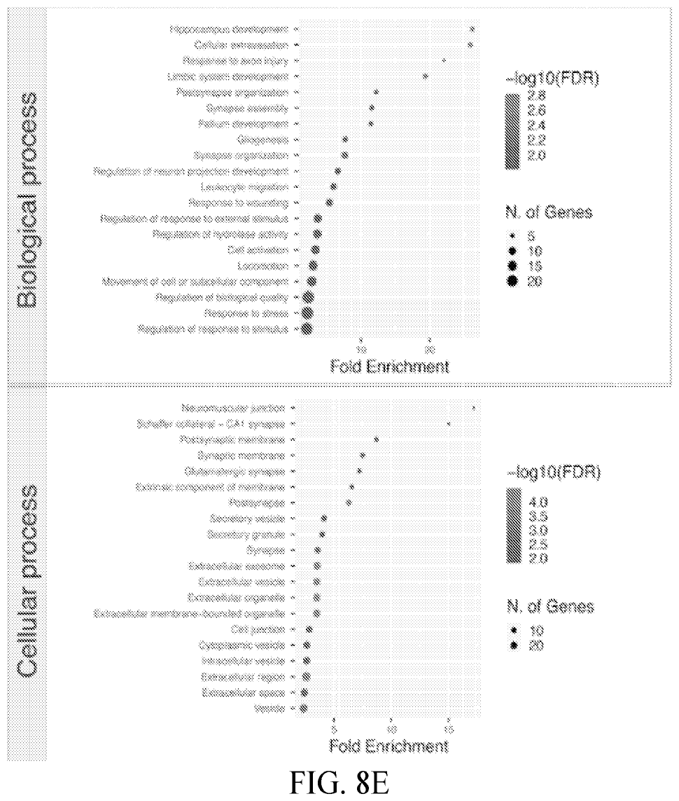

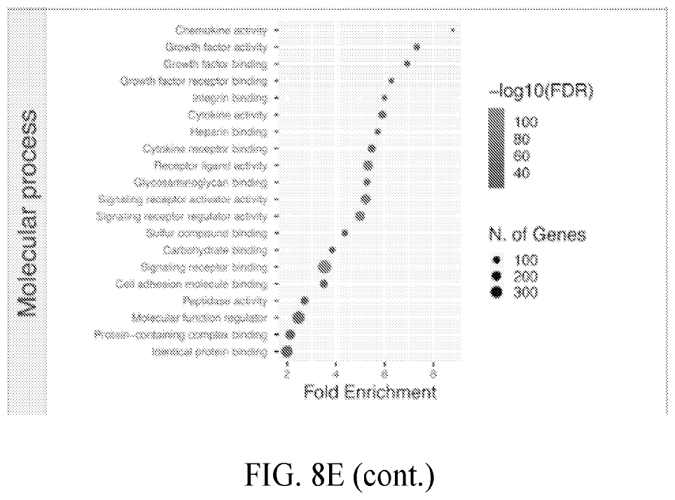

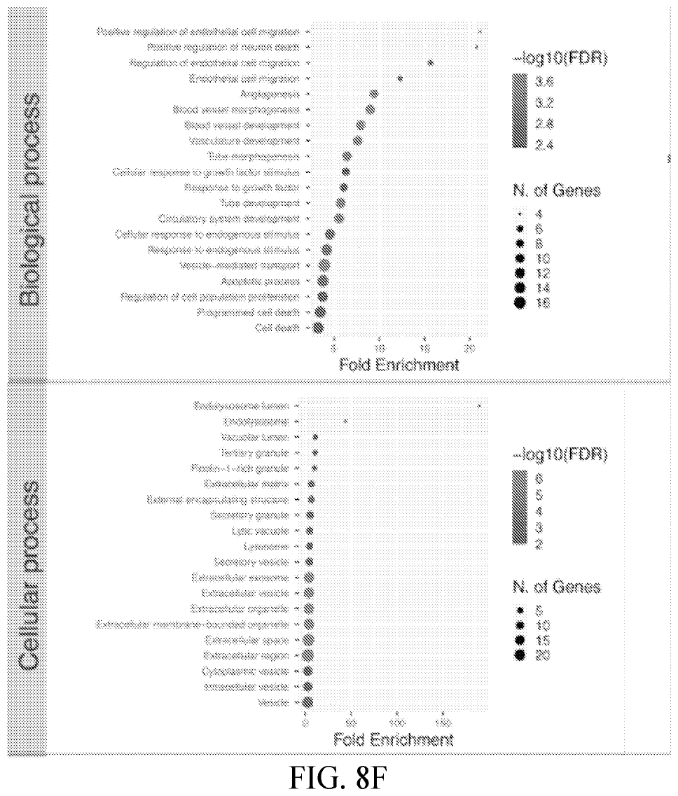

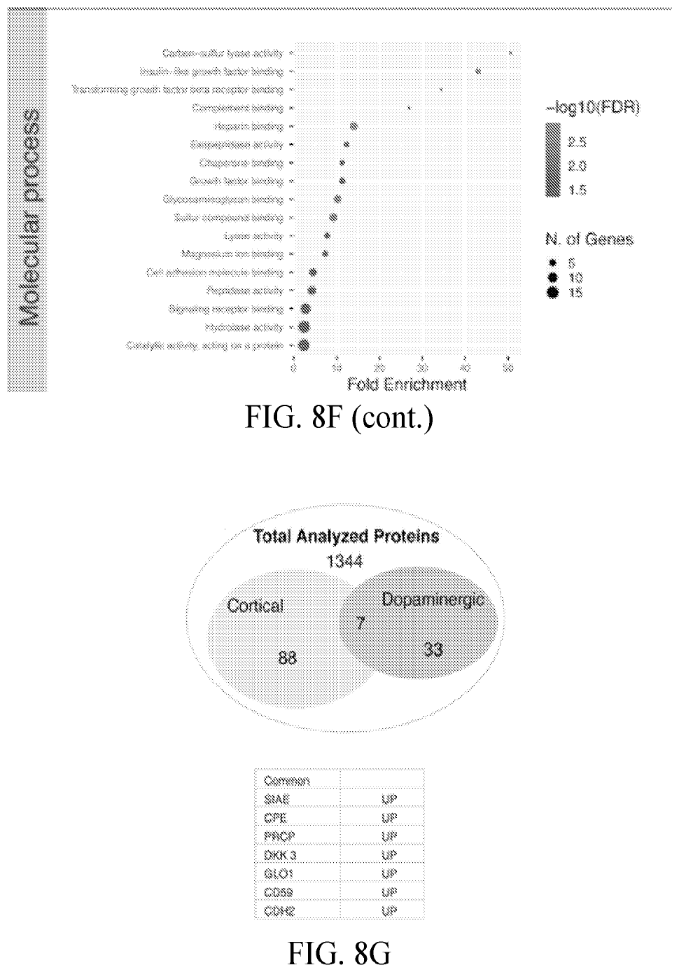

Microgravity influences protein secretion. To assess the effects of microgravity on secretion of proteins, culture medium was collected from vials containing single organoids and analyzed using Olink® Target 1536. All of the organoid types showed LEO vs ground differentially secreted proteins (DSPs) (padj<0.05, Welch two-sample t-test) (FIGS. 8A-8G). 40 DSPs were identified in the medium from dopamine neuron organoids and 95 from cortical organoid medium. Seven proteins were found to be secreted at higher levels in LEO by both dopaminergic and cortical samples (Table 3).

TABLE 3
Differentially secreted proteins (DSPs) with
higher levels in Low Earth Orbit (LEO).

| Differentially secreted proteins | Location/Function |
|---|---|
| sialic acid acetylesterase (SIAE) | cytosol, lysosome |
| carboxypeptidase E (CPE) | peripheral membrane protein |
| prolylcarboxypeptidase (PRCP) | lysosomal |
| dickkopf WNT signaling pathway | secreted protein, morphogen |
| inhibitor 3 (DKK3) | |
| glyoxalase I (GLO1) | cytosol |
| CD59 | cell surface glycoprotein |
| cadherin 2 CDH2 | cell surface glycoprotein |

In summary, the above-described study and experiments is a first-in-kind ISS study on neuroinflammation using brain organoids derived from iPSC lines from people with PD and MS. This pioneering 3D human model system in LEO established that it was possible to maintain long-term (at least one month) cultures of iPSC-derived CNS cells, and suggested previously unstudied effects of microgravity on the brain. The foundational work reported here provides a novel human model to improve the understanding of the perturbations underlying neuroinflammation and neurodegeneration both in space and here on Earth.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Yao Z, Mich J K, Ku S, et al (2017) A Single-Cell Roadmap of Lineage Bifurcation in Human ESC Models of Embryonic Brain Development. Cell Stem Cell 20:120-134.
2. Kriks S, Shim J-W, Piao J, et al (2011) Dopamine neurons derived from human E S cells efficiently engraft in animal models of Parkinson's disease. Nature 480:547-551.
3. Douvaras P, Sun B, Wang M, et al (2017) Directed Differentiation of Human Pluripotent Stem Cells to Microglia. Stem Cell Rep.
4. Afshinnekoo E, Scott R T, MacKay M J, et al (2020) Fundamental Biological Features of Spaceflight: Advancing the Field to Enable Deep-Space Exploration. Cell 183:1162-1184
5. White R J, Averner M (2001) Humans in space. Nature 409:1115-1118.
6. Berrone E, Cardone F, Corona C, et al (2020) The Amyloid Aggregation Study on Board the International Space Station, an Update. Aerotec Missili Spaz 99:141-148.
7. Hargens A R, Vico L (2016) Long-duration bed rest as an analog to microgravity. J Appl Physiol 120:891-903.
8. Sarkar P, Sarkar S, Ramesh V, et al (2006) Proteomic analysis of mice hippocampus in simulated microgravity environment. J Proteome Res 5:548-553.
9. Reschke M F, Good E F, Clement G R (2017) Neurovestibular Symptoms in Astronauts Immediately after Space Shuttle and International Space Station Missions. OTO Open 1:2473974X17738767.
10. Garrett-Bakelman F E, Darshi M, Green S J, et al (2019) The NASA twins study: A multidimensional analysis of a year-long human spaceflight. Science 364.
11. Crucian B E, Zwart S R, Mehta S, et al (2014) Plasma cytokine concentrations indicate that in vivo hormonal regulation of immunity is altered during long-duration spaceflight. J Interferon Cytokine Res 34:778-786.
12. 2021 Alzheimer's disease facts and figures. Alzheimer's Dement J Alzheimer's Assoc (2021) 17:327-406. https://doi.org/10.1002/alz.12328
13. Mulica P, Grunewald A, Pereira S L (2021) Astrocyte-Neuron Metabolic Crosstalk in Neurodegeneration: A Mitochondrial Perspective. Front Endocrinol 12:668517.
14. Muzio L, Viotti A, Martino G (2021) Microglia in Neuroinflammation and Neurodegeneration: From Understanding to Therapy. Front Neurosci 15:742065.
15. Crawford-Young S J (2006) Effects of microgravity on cell cytoskeleton and embryogenesis. Int J Dev Biol 50:183-191.
16. Koch M W, Cutter G, Stys P K, et al (2013) Treatment trials in progressive MS—current challenges and future directions. Nat Rev Neurol 9:496-503.
17. Takahashi K, Yamanaka S (2006) Induction of Pluripotent Stem Cells from Mouse Embryonic and Adult Fibroblast Cultures by Defined Factors. Cell 126:663-676.
18. Madhavan M, Nevin Z S, Shick H E, et al (2018) Induction of myelinating oligodendrocytes in human cortical spheroids. Nat Methods.
19. Stachowiak E K, Benson C A, Narla S T, et al (2017) Cerebral organoids reveal early cortical maldevelopment in schizophrenia-computational anatomy and genomics, role of FGFR1. Transl Psychiatry 7:6.
20. Mariani J, Coppola G, Zhang P, et al (2015) FOXG1-Dependent Dysregulation of GABA/Glutamate Neuron Differentiation in Autism Spectrum Disorders. Cell 162:375-390.
21. Douvaras P, Sun B, Wang M, et al (2017) Directed Differentiation of Human Pluripotent Stem Cells to Microglia. Stem Cell Rep 8:1516-1524.
22. Douvaras P, Wang J, Zimmer M, et al (2014) Efficient generation of myelinating oligodendrocytes from primary progressive multiple sclerosis patients by induced pluripotent stem cells. Stem Cell Rep 3:250-9.
23. Fattorelli N, Martinez-Muriana A, Wolfs L, et al (2021) Stem-cell-derived human microglia transplanted into mouse brain to study human disease. Nat Protoc.
24. Kriks S, Shim J-W, Piao J, et al (2011) Dopamine neurons derived from human E S cells efficiently engraft in animal models of Parkinson's disease. Nature 480:547-551.
25. Yao Z, Mich J K, Ku S, et al (2017) A Single-Cell Roadmap of Lineage Bifurcation in Human ESC Models of Embryonic Brain Development. Cell Stem Cell 20:120-134.
26. Paull D, Sevilla A, Zhou H, et al (2015) Automated, high-throughput derivation, characterization and differentiation of induced pluripotent stem cells. Nat Methods 12:885-892.
27. Shimura N, Kojima S (2018) The Lowest Radiation Dose Having Molecular Changes in the Living Body. Dose-Response Publ Int Hormesis Soc 16:1559325818777326.
28. Sokolov M, Neumann R (2014) Effects of low doses of ionizing radiation exposures on stress-responsive gene expression in human embryonic stem cells. Int J Mol Sci 15:588-604.
29. Sloan S A, Darmanis S, Huber N, et al (2017) Human Astrocyte Maturation Captured in 3D Cerebral Cortical Spheroids Derived from Pluripotent Stem Cells. Neuron 95:779-790.e6.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of culturing an organoid, comprising:
combining a neural precursor cell with a first volume of a cortical media or a dopaminergic media to form an organoid;
adding a microglia to the organoid;
adding the combined organoid and iPSC-derived microglia to a cryovial with a second volume of the cortical media or the dopaminergic media, the second volume of the cortical media or the dopaminergic media including an amount of interleukin (IL)-34 and granulocyte-macrophage colony-stimulating factor (GM-CSF) and further including an amount of a buffering solution;
sealing the cryovial; and
culturing the organoid for a pre-determined period of time under closed conditions.

2. The method of claim 1, wherein the neural precursor cell, the microglia, or both are derived from a neural stem cell.

3. The method of claim 2, wherein the neural stem cell is an iPSC.

4. The method of claim 2, wherein the neural precursor cell is obtained from a healthy subject or a subject having a neurodegenerative disease.

5. The method of claim 4, wherein the subject has a neurodegenerative disease, and wherein the neurodegenerative disease is selected from Multiple Sclerosis and Parkinson's disease.

6. The method of claim 1, wherein combining the neural precursor cell with the first volume of a cortical media or a dopaminergic media comprises combining the neural precursor cell with cortical media, and wherein the cortical media includes an effective amount of cyclic adenosine monophosphate (cAMP), brain-derived neurotrophic factor (BDNF), glial cell-derived neurotrophic factor (GDNF), and neurotrophin (NT)-3.

7. The method of claim 6, wherein the cAMP is included at a concentration of about 100 mM, the BDNF is included at a concentration of about 10 ng/ml, the GDNF is included at a concentration of about 10 ng/ml, and the NT-3 is included at a concentration of about 10 ng/ml.

8. The method of claim 6, wherein the IL-34 is included at a concentration of about 100 ng/ml and the GM-CSF is included at a concentration of about 10 ng/ml.

9. The method of 1, wherein combining the neural precursor cell with the first volume of a cortical media or a dopaminergic media comprises combining the neural precursor cell with dopaminergic media, and wherein the dopaminergic media includes an effective amount of BDNF, GDNF, transforming growth factor (TGF)-03, ascorbic acid, and cAMP.

10. The method of claim 9, wherein the BDNF is included at a concentration of about 20 ng/ml, the GDNF is included at a concentration of about 20 ng/ml, the TGF-β3 is included at a concentration of about 1 ng/ml, the ascorbic acid is included at a concentration of about 200 μM, and the cAMP is included at a concentration of about 500 μM.

11. The method of claim 9, wherein the IL-34 is included at a concentration of about 100 ng/ml and the GM-CSF is included at a concentration of about 10 ng/ml.

12. The method of claim 1, wherein culturing the cells for a predetermined period of time comprises culturing the cells for the predetermined period of time under microgravity conditions.

13. The method of claim 1, wherein the pre-determined period of time is at least 28 days.

14. The method of claim 13, further comprising the steps of:
removing the organoid from the cryovial after the predetermined time period; and
culturing the organoid in a tissue culture vessel.

15. The method of claim 1, wherein the organoid comprises a single organoid.

16. A system for culturing an organoid, comprising:
an organoid formed from a neural precursor cell;
a microglia;
an effective amount of a cortical media or a dopaminergic media;
a buffering solution; and
a cryovial for housing the organoid, the microglia, the cortical media or the dopaminergic media, and the buffering solution under closed conditions.

17. The system of claim 16, wherein the neural precursor cell, the microglia, or both are derived from a neural stem cell.

18. The system of claim 17, wherein the neural stem cell is an iPSC.

19. The system of claim 18, wherein the iPSC is obtained from a healthy subject or a subject having a neurodegenerative disease.

20. The system of claim 19, wherein the subject has a neurodegenerative disease, and wherein the neurodegenerative disease is selected from Multiple Sclerosis and Parkinson's disease.

21. The system of claim 16, wherein the cortical media includes an effective amount of cAMP, BDNF, GDNF, and NT-3.

22. The system of claim 20, wherein the cAMP in included at a concentration of about 100 mM, the BDNF is included at a concentration of about 10 ng/ml, the GDNF is included at a concentration of about 10 ng/ml, and the NT-3 is included at a concentration of about 10 ng/ml.

23. The system of claim 16, wherein the dopaminergic media includes an effective amount of BDNF, GDNF, TGF-β3, ascorbic acid, and cAMP.

24. The system of claim 23, wherein the BDNF is included at a concentration of about 20 ng/ml, the GDNF is included at a concentration of about 20 ng/ml, the TGF-β3 is included at a concentration of about 1 ng/ml, the ascorbic acid is included at a concentration of about 200 μM, and the cAMP in included at a concentration of about 500 μM.

25. The system of claim 16, wherein the cortical media or the dopaminergic media comprises an amount of IL-34 and GM-CSF.

26. The system of claim 25, wherein the IL-34 is included at a concentration of about 100 ng/ml and the GM-CSF is included at a concentration of about 10 ng/ml.
